(19) United States
(12) Patent Application Publication
(10) Pub. No.: US 2005/0043599 A1
(43) Pub. Date: Feb. 24, 2005

(54) Pulse oximetry device and method

(76) Inventor: Sean T. O'Mara, San Antonio, TX (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/475,200

(22) PCT Filed: Apr. 18, 2002
(86) PCT No.: PCT/US02/12914

(30) Foreign Application Priority Data
Apr. 19, 2001 (US) 60284834

(51) Int. Cl. A61B 5/00

(52) U.S. Cl. 600/344; 600/339

(57) ABSTRACT

In one embodiment, an anal pulse oximeter device is characterized by an anal canal surface; and a rectal-vault cuff having a leading portion and a trailing portion, the trailing portion substantially proximate to said anal canal surface. In another embodiment, a method of using a pulse oximeter device is characterized by positioning an insertion end of the pulse oximeter device against an anus; advancing the pulse oximeter device until a rectal-vault cuff of the pulse oximeter device substantially clears an internal anal sphincter; and withdrawing the pulse oximeter device until the rectal-vault cuff of the pulse oximeter device contacts a rectal vault. In another embodiment, a method of manufacturing a pulse oximeter device is characterized by attaching a rectal-vault cuff, the rectal-vault cuff having a leading portion and a trailing portion, such that the trailing portion is substantially proximate to an anal canal surface. In various embodiments the anal canal surface is an anatomical anal canal surface, while in other embodiments the anal canal surface is a surgical anal canal surface.

### CROSS-REFERENCE TO RELATED APPLICATION

This application claims the benefit under 35 U.S.C. § 119(e) of U.S. Provisional Patent Application No. 60/284,834 filed Apr. 19, 2001, entitled Anal Pulse Oximetry Monitoring Device, naming Sean T. O'Mara as inventor, said provisional patent application hereby incorporated by reference, in its entirety, into the detailed description portion of the present application. The incorporated-by-reference provisional patent application has been incorporated into the detailed description portion of the present application because the incorporated-by-reference provisional patent application described aspects of both the related art and the present patent application under a “background information” section; however, the description of aspects of the present patent application under the “background information” section of the provisional patent application is in no way an admission that such related art or aspects of the present invention constituted “prior art”. In fact, several aspects of the present patent application predate the related-art aspects described in the provisional patent application. Accordingly, the foregoing statements constitute public notice that the provisional patent application was intended to contain no admissions related to prior art whatsoever.

### STATEMENT REGARDING GOVERNMENT INTEREST

The State of Texas has certain rights in this invention.

### BACKGROUND OF THE INVENTION

1. Field of the Invention

The present invention relates, in general, to pulse oximetry devices and methods.

2. Description of the Related Art

Pulse oximetry refers to the process of inferring the oxygen-hemoglobin saturation of a patient's blood via use of a photoelectric oximeter.

It has long been known in the art how to correlate the reflectance or transmittance of certain wavelengths of light (e.g., light having wavelengths which constitute visible red light and/or light having wavelengths that constitute infrared light) with the oxygen content (or oxygen saturation) of pulsing blood. Consequently, in one type of pulse oximetry, known in the art as reflectance pulse oximetry, a light source, such as a light emitting diode (LED) and a light sensor, such as a photosensor (e.g., a photodiode), are positioned to one side of a portion a patient's circulatory system. Thereafter, the LED is activated, and the photosensor is monitored to collect data on light reflected from the portion of the patient's circulatory system. Using algorithms well known to those within the art, the amount of oxygen saturation of the patient's blood is then inferred based on the measured reflectance data. For example, in one implementation of reflectance pulse oximetry, both the LED and the photosensor are positioned against a patient's tympanic membrane (i.e., in the ear), and measured reflected light is used to infer the oxygen saturation of the patient.

In another type of pulse oximetry, known in the art as transmittal pulse oximetry, a light emitting diode (LED) and a photosensor (e.g., a photodiode) are positioned on either side of a portion a patient's circulatory system. Thereafter, the LED is activated, and the photosensor is monitored to collect data on light transmitted through the portion of the patient's circulatory system. Using algorithms well known to those within the art, the amount of oxygen saturation of the patient's blood is then inferred based on the measured transmittance data. For example, in one well-known implementation of transmittal pulse oximetry, the LED and the photosensor are positioned on either side of a patient's finger via use of a finger clip, and measured transmitted light is used to infer the oxygen saturation of the patient.

### BRIEF SUMMARY OF THE INVENTION

The inventor named herein has devised a pulse oximetry device and method.

In one embodiment, an anal pulse oximeter device is characterized by an anal canal surface; and a rectal-vault cuff having a leading portion and a trailing portion, the trailing portion substantially proximate to said anal canal surface.

In another embodiment, a method of using a pulse oximeter device is characterized by positioning an insertion end of the pulse oximeter device against an anus; advancing the pulse oximeter device until a rectal-vault cuff of the pulse oximeter device substantially clears an internal anal sphincter; and withdrawing the pulse oximeter device until the rectal-vault cuff of the pulse oximeter device contacts a rectal vault.

In another embodiment, a method of manufacturing a pulse oximeter device is characterized by attaching a rectal-vault cuff, the rectal-vault cuff having a leading portion and a trailing portion, such that the trailing portion is substantially proximate to an anal canal surface.

In various embodiments the anal canal surface is an anatomical anal canal surface, while in other embodiments the anal canal surface is a surgical anal canal surface.

The foregoing is a summary and thus contains, by necessity, simplifications, generalizations and omissions of detail; consequently, those skilled in the art will appreciate that the summary is illustrative only and is NOT intended to be in any way limiting. Other aspects, inventive features, and advantages of the devices and/or processes described herein, as defined solely by the claims, will become apparent in the non-limiting detailed description set forth herein.

### BRIEF DESCRIPTION OF THE SEVERAL VIEWS OF THE DRAWING(S)

FIG. 1 shows a perspective view of a pulse oximeter device 100.

FIG. 2 depicts a side-plan view of the pulse oximeter device 100 anatomically positioned in anatomical structures of a patient's body 200

FIG. 3 depicts a side-plan view of an alternate implementation of the pulse oximeter device 100, which is particularly useful to novice health-care providers.

The use of the same symbols in different drawings typically indicates similar or identical items.

### DETAILED DESCRIPTION OF THE INVENTION

The inventor named herein (the inventor) has devised a device and related process which treat what is ordinarily viewed in the related art as a “barrier” to a preferable location for pulse oximetry as a near optimal location for pulse oximetry. Specifically, the inventor named herein has devised a device and related process which utilize at least a part of the anatomical/surgical anal canal as a preferable location for pulse oximetry. Those having ordinary skill in the art will appreciate that, as used herein, the term “anatomical/surgical anal canal” is meant to encompass either or both the anatomical anal canal and the surgical anal canal. Those skilled in the art will recognize that the anatomical anal canal is typically understood to extend from what is known in the art as the anal verge to what is known in the art as the dentate line, and is generally about 1.5 cm long in an adult human male. Those skilled in the art will also recognize that the surgical anal canal is typically understood to extend from what is known in the art as the anal verge to the anorectal ring/puborectalis muscle, is typically about 3-4 cm's in length in an adult human male, and ends where the anatomical rectum begins.

The inventor often works as an emergency room physician. Consequently, the inventor has had occasion to work with many patients in shock. The inventor has recognized that for patients in shock many related art pulse oximetry devices and processes do not work well. Specifically, the inventor has recognized that for patients in shock (e.g., patients in traumatic, septic, hypovolemic, or neurogenic shock) the patients' bodies tend to decrease the amount blood allowed to circulate to the patients' extremities (e.g., hands, fingers, feet, or toes), in preference for the blood allowed to circulate within the core (e.g., head and thoracic cavity) of the patients' bodies.

The inventor has recognized that, insofar as that shock patients' bodies tend to decrease the amount of blood allowed to circulate to the extremities, noninvasive pulse oximetry devices (e.g., those placed on the finger, so, or earlobe of the patient) tend to give inaccurate results. Accordingly, the inventor has hypothesized that it would be preferable to obtain pulse oximetry measurements near to the core of patients' bodies in order to get more accurate pulse oximetry measurements.

In the course of his work as an emergency room physician, the inventor has unfortunately had occasion to observe several patients dying. The inventor has noticed that when patients die, one of the very last body systems to fail is the voluntary/involuntary control of the anatomical/surgical anal canal. That is, the inventor has noticed that when a patient is terminal, the patient's body will tend to maintain voluntary/involuntary control of the anatomical/surgical anal canal until substantially immediately (e.g., two or three minutes) before death.

The inventor has noticed that the anatomical/surgical anal canal, which includes the internal and external anal sphincters, has some of the highest muscle tone in the human body. The inventor has further recognized, based on physiological principles, that in order for the human body to maintain such muscle tone the human body must supply the muscles of the anatomical/surgical anal canal with a high degree of oxygen, through rich perfusion of blood, in order for such muscles to continue to function. Insofar as the inventor has observed that the human body tends to maintain the functioning of the anatomical/surgical anal canal until immediately preceding death, the inventor has hypothesized that the human body will tend to maintain circulation to the muscles of the anatomical/surgical anal canal, even during a state of physiological shock.

The inventor has noticed that the distal walls of the anatomical/surgical anal canal tend to be relatively “clean” (e.g., not contaminated with large degrees fecal matter such as are normally present in the rectum). As noted, the inventor has also hypothesized that the human body will tend to maintain good circulation to the anatomicausurgical anal canal up to almost the time of clinical death. Consequently, the inventor has hypothesized that the anatomical/surgical anal canal constitutes a particularly good area from which to obtain pulse oximetry data.

While the inventor has hypothesized that the anatomical/surgical anal canal would constitute a particularly good area from which to obtain pulse oximetry data, the inventor has also recognized that the anatomical/surgical anal canal is also highly susceptible to injury. That is, the inventor has recognized that the high degree of, and need for, vascularization within the anatomical/surgical anal canal renders the anatomical/surgical anal canal highly susceptible to pressure necrosis (e.g., cell death due to continuous pressure in the same region of the anatomical/surgical anal canal over an extended period of time). In addition, the inventor has noticed that the anatomical/surgical anal canal is also highly innervated, and consequently patients tend to complain of discomfort when devices having significant protrusions are present within the anatomical/surgical anal canal. Accordingly, the inventor has hypothesized that a device for the collection of pulse oximetry data from the anatomical/surgical anal canal should preferably not have significant protrusions, or lobes, along the length of the device which tends to be resident within the anatomical/surgical anal canal, so as to avoid the possibility of significant pressure necrosis or patient discomfort.

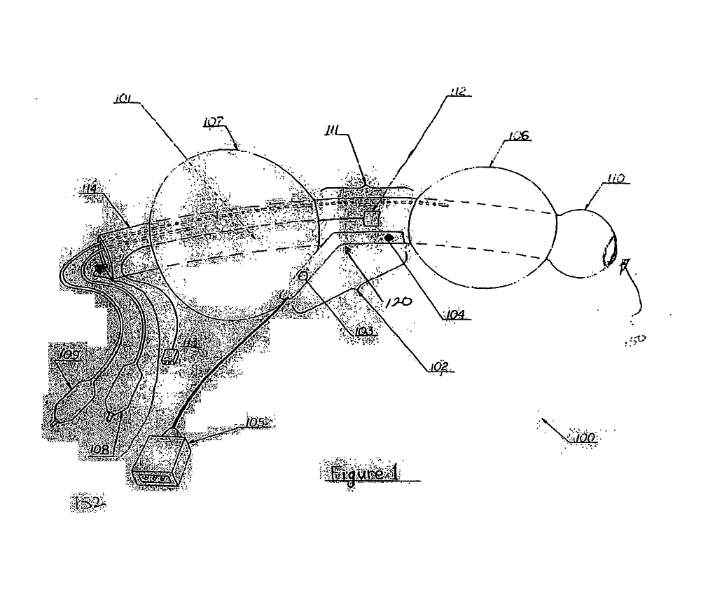

With reference to the figures, and with reference now to FIG. 1, shown is a perspective view of a pulse oximeter device 100. Depicted is that in one embodiment the pulse oximeter device 100 has a spherical or rounded insertion end 110, which in one embodiment is larger in diameter than the diameter of pulse oximeter device's cylindrical body 101. In one embodiment, the rounded insertion end 110 is rounded to decrease the likelihood of perforation of, or other forms of traumatic injury to, the patient's anatomical/surgical anal canal, rectum, or other regionally related anatomy during placement of the pulse oximeter device 100. Those having ordinary skill in the art will appreciate that typically, the larger the diameter of the rounded insertion end 110, the more discomfort a patient will experience upon placement of the pulse oximeter device 100. However, those having ordinary skill in the art will also appreciate that the smaller the diameter of the rounded insertion end 110, the more likely it is that a perforation, or other form of injury, will occur. Consequently, the exact diameter of the rounded insertion end 110 is a design choice within the purview of the device designer. In one implementation, the rounded insertion end 110 is only slightly wider (e.g., 1 mm) than the diameter of the pulse oximeter device's cylindrical body 101, while in other embodiment the rounded insertion end 110 is significantly wider (e.g., 5 mm) than the pulse oximeter device's cylindrical body 101.

In operation, after a suitable lubricant has been effectively administered, the rounded insertion end 110 of the pulse oximeter device 100 is carefully positioned against a patient's external anus, and gentle pressure applied, in order to advance the rounded insertion end 110 through the patient's anatomical/surgical anal canal until the rectal-vault inflatable cuff 106 has substantially cleared the patient's anatomical/surgical anal canal to substantially reside within the patient's rectum. In one implementation this is achieved via the health-care provider digitally ensuring that the rectal-vault inflatable cuff 106 is so placed; in other words, the health-care provider inserts his finger along the pulse oximeter device's cylindrical body 101 and ensures, by feel, that the rectal-vault inflatable cuff 106 has substantially cleared the patient's anatomical/surgical anal canal (e.g. has substantially cleared the internal anal sphincter).

Subsequent to the rectal-vault inflatable cuff 106 substantially clearing the patient's anatomical/surgical anal canal, the rectal-vault inflatable cuff 106 is typically inflated with a liquid medium such as standard saline. In another implementation, a gas, such as air, is used to inflate the rectal-vault inflatable cuff 106. In one implementation, the inflation is achieved via access port 109, which is constructed to allow inflation via a standard syringe. The inventor points out that, due to the ways in which inflatable cuffs tend to operate, those having ordinary skill in the art will appreciate that even if a small portion of the rectal-vault inflatable cuff 106 still resides in a patient's anatomical/surgical anal canal upon initial inflation, the portion of the rectal-vault inflatable cuff 106 which resides in the rectal vault will tend to inflate first (that portion not being under the pressure of the internal anal sphincter), and hence substantially pull the remainder of the rectal-vault inflatable cuff 106 into its desired position within the rectal vault of the patient. That is, in one implementation, upon inflation, the operation of the rectal-vault inflatable cuff 106 helps to effectively position the rectal-vault inflatable cuff 106 such that anatomical/surgical anal canal surface 111 tends to substantially reside in the patient's anatomical/surgical anal canal and such that the rectal-vault inflatable cuff 106 tends to substantially reside outside the patient's anatomical/surgical anal canal and inside the patient's rectal vault.

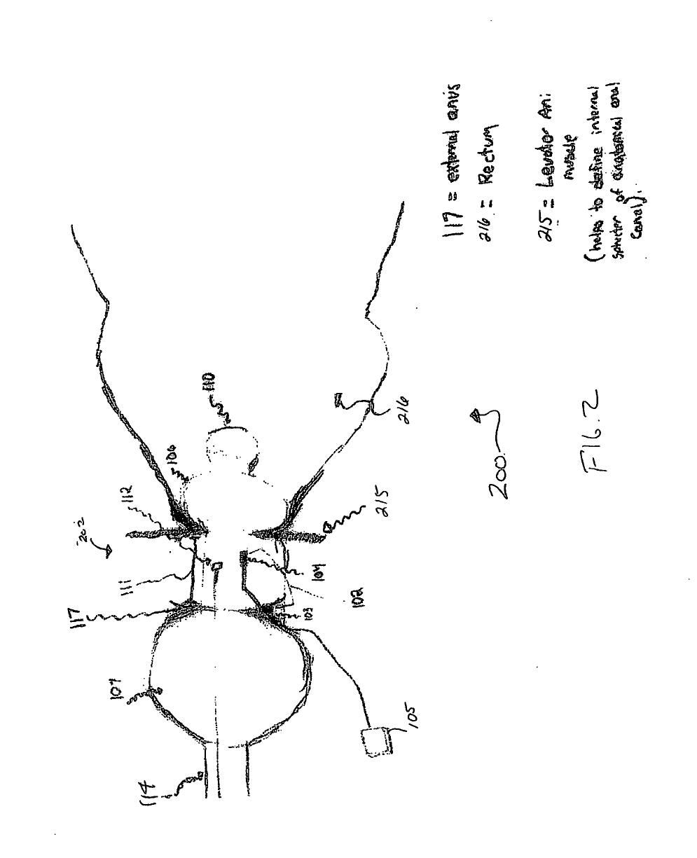

Following the inflation of the rectal-vault inflatable cuff 106, typically the health-care provider will apply gentle pressure in the direction opposite that used to insert the pulse oximeter device 100 in order to ensure that the inflated rectal-vault inflatable cuff 106 is in gentle contact with at least a part of the patient's rectal vault (e.g., in proximity to the levator ani muscle of FIG. 2, and the pubic rectalis/anal rectal ring which those having ordinary skill in the art will recognize tends to define the boundary between the patient's rectum and the anatomical/surgical anal canal). In one implementation, the rectal positioning of the inflated rectal-vault inflatable cuff 106 is achieved by the health-care provider applying pressure until he tactilely determines that the inflated rectal-vault inflatable cuff 106 is in contact with at least part of the patient's rectal vault. In another implementation, the rectal positioning of the inflated rectal-vault inflatable cuff 106 is achieved by the health-care provider applying pressure until the patient orally confirms that he feels a sensation of contact (e.g., a pulling, or tugging sensation) in his anal area and/or the health care provider experiences resistance to gentle retraction of the inflated cuffed device.

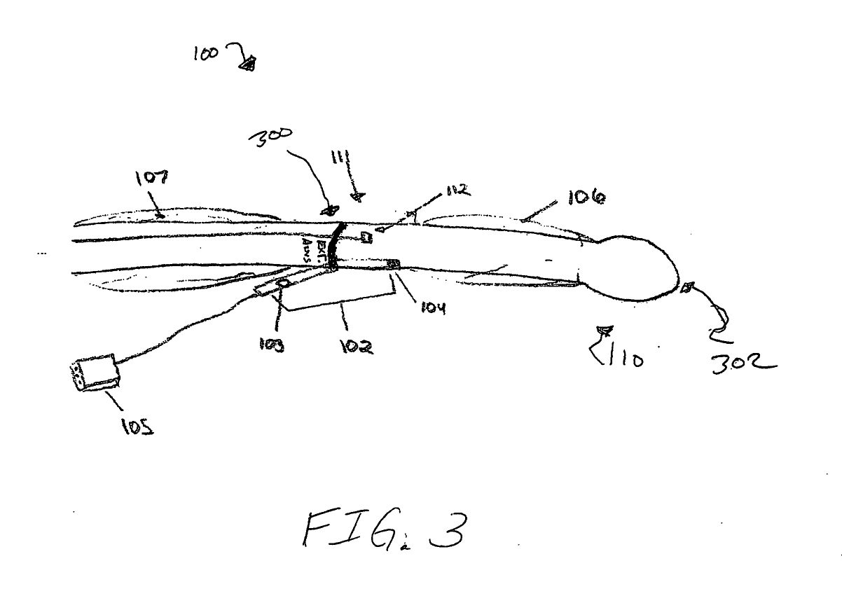

Continuing to refer to FIG. 1, shown is that anatomical/surgical anal canal surface 111 of the pulse oximeter device 100, has substantially flush with its inferior external surface (with the patient laying down in the supine position), a physiological sensor 112 to measure vital signs of the patient (e.g., a temperature physiological sensor to measure the core body temperature of the patient, a manometer physiological sensor to measure the pressure of the patient's anatomical/surgical anal canal, or other types of sensors to measure various physiological functions). Those having ordinary skill in the art will appreciate that while only one physiological sensor 112 is shown for ease of presentation, physiological sensor 112 is intended to be representative of one or more physiological sensors substantially flush with the anatomical/surgical anal canal surface 111 of the pulse oximeter device 100. Depicted is that the pulse oximeter device 100 has affixed, substantially flush with its external posterior surface, an implementation of a pulse oximetry assembly 102, which, when positioned as described herein, allows for sampling of the oxygen saturation of the blood perfusing the vascularly rich anal canal mucosa. In one implementation, the pulse oximetry assembly 102 is shorter than that depicted in FIG. 1, such that the pulse oximetry assembly 102 resides substantially between the confines of external inflatable cuff 107 and rectal-vault inflatable cuff 106. In such implementation, the photosensor 103 and the LED 104 are in substantially the same plane, and reflectance pulse oximetry is used. However, in the implementation shown in FIG. 1, the pulse oximetry assembly 102 is of length sufficient to substantially overlap external inflatable cuff 107 when external inflatable cuff 107 is uninflated, (e.g., as shown in FIG. 3).

In operation, once the pulse oximeter device 100 has been positioned such that the inflated rectal-vault inflatable cuff 106 is seated against at least part of the patient's rectal vault, the external inflatable cuff 107 is typically inflated with a liquid medium such as standard saline. In another implementation, a gas, such as air, is used to inflate the rectal-vault inflatable cuff 107. In one implementation, the inflation is achieved via an access port 108, which is constructed to allow inflation via a standard syringe. As shown in FIG. 1 (and FIG. 2), such inflation of the external inflatable cuff 107 causes the pulse oximetry assembly 102 to bend, or flex, around its pivot point 120 such that the photosensor 103 is positioned to receive light transmitted by the LED 104 substantially by transmittance rather than substantially by reflectance. In one implementation this is achieved via orienting the LED 104 such that its main axis of transmission is substantially in line with an expected position of the photosensor 103. In one implementation, the photosensor 103 is angled such that its main axis of reception will be substantially in line with the main axis of transmission of the LED 104 when the external inflatable cuff 107 is inflated. Those having ordinary skill in the art will appreciate that positions of the axes of transmission and reception of the LED 104 and the photosensor 103 will necessarily be approximate, and will depend upon the expected patient population. It is expected that such positioning will typically be determined empirically. In one implementation, the main axes of transmission and reception of the LED 104 and the photosensor 103 are positioned at 45 degrees relative to the surface of the pulse oximetry assembly 102 spanning the distance between the LED 104 and the photosensor 103. Those having ordinary skill in the art will also recognize that the relative positions of the photosensor 103 and the LED 104 can be relatively easily reversed via a minimal amount of experimentation well within the ambit of one having ordinary skill in the art; that is, the photosensor 103 could be placed in the positions in which the LED 104 is shown and/or described herein, and the LED 104 could be placed in the positions in which the photosensor 103 is shown and/or described herein, and the pulse oximeter device 100 would still effectively function in substantially the fashion shown and/or described herein.

Subsequent to the external inflatable cuff 107 being inflated, data may be collected via use of the LED 104 and the photosensor 103 via the pulse oximetry assembly's 102 adapter/plug 105, which can be attached to any universal prong pulse oximeter for data interpretation and waveform production. The patient's recorded data from the physiological sensor 112 is similarly conducted down and out the physiological sensor adapter/plug 113 for digital display from a variety of display devices.

In addition to the foregoing, those having ordinary skill in the art will appreciate that many times, and especially for patients in intensive care or operating room settings, patients often have several bowel movements a day. Accordingly, in one implementation, the pulse oximeter device 100 includes a bowel-evacuation channel (e.g., hollow inner channel) through which stool from a patient's bowel may be evacuated. Accordingly, shown in FIG. 1 is that, in one implementation, the rounded insertion end 110 has an open hole 150 which forms a part of hollow tube 152 enclosed by the surface of the pulse oximeter device 100. Those skilled in art will appreciate that, when pulse oximeter device 100 is positioned within a patient's anatomical/surgical anal canal, the presence of open hole 150 which forms a part of hollow tube 152 will allow staff to quickly and effectively evacuate a patient's bowel.

Referring now to FIG. 2, depicted is a side-plan view of the pulse oximeter device 100 anatomically positioned in anatomical structures of a patient's body 200. Illustrated is that the rectal-vault inflatable cuff 106 is inflated within the rectum 216 and positioned against at least part of the rectal vault (e.g., proximate to the levator ani muscle and the pubic rectalis/rectal ring 215 which helps to anatomically define the boundary between internal anal sphincter of the anatomicausurgical anal canal and the rectal vault). Further illustrated is that the external inflatable cuff 107 is inflated outside the patient's body 200. As can be seen, the anatomical/surgical anal canal surface 111 is formed substantially smoothly (e.g., without significant protrusions), such that pressure is relatively evenly applied within the anatomical/surgical anal canal 202, thereby decreasing the likelihood of injury due to unique pressure points within the anatomical/surgical anal canal 202 (e.g., is formed in a roughly cylindrical shape). As can further be seen, the inflated external inflatable cuff 107 is constructed to position the photosensor 103 in the proximity of the external anus 117, such that transmittance oximetry may be utilized.

With reference now to FIG. 3, depicted is a side-plan view of an alternate implementation of the pulse oximeter device 100, which is particularly useful to novice health-care providers. Illustrated is the alternate pulse oximeter device 100 having “external anus” labeled striping 300. In one implementation, the “external anus” labeled striping 300 is located on the pulse oximeter device 100 at a position appropriate to the average anatomical/surgical anal canal length of the average adult male human, such that if the health care provider inserts the pulse oximeter device 100 to the point such that at least the leading edge of the “external anus” labeled striping 300 contacts an average adult patient's external anus, there is a high likelihood that rectal-vault inflatable cuff 106 has substantially cleared the anatomical/surgical anal canal of the patient. In one implementation, the leading edge of the “external anus” labeled striping 300 is located approximately 6 centimeters from the leading surface 302 of the rounded insertion end 110, since the anatomical/surgical anal canal of an adult human male typically ranges from 3-5 centimeters. Exactly where the leading edge of the “external anus” labeled striping 300 will be placed relative to the leading surface 302 of the rounded insertion end 110 will depend upon an the expected patient population, and the length of the rectal-vault inflatable cuff 106. The position of the leading surface 302 of the rounded insertion end 110 and the length of the rectal-vault inflatable cuff 106 are typically design choices within the purview of the system designer.

Continuing to refer to FIG. 3, illustrated is that, in one implementation, when the external inflatable cuff 107 is deflated, the pulse oximetry assembly 102 lies against the external inflatable cuff 107, and thus the photosensor 103 is in substantially the same plane as the LED 104. As has been shown and described (e.g., in relation to FIGS. 1 and 2), in one implementation, upon inflation, the external inflatable cuff 107 causes the pulse oximetry assembly 102 to bend about its pivot point 120, such that the photosensor 103 is ultimately forced into a position to allow for transmittance oximetry. As has also been discussed, in another implementation the pulse oximetry assembly 102 resides within the confines of external cuff 107 and rectal-vault inflatable cuff 106 in order to allow for reflectance oximetry. In yet another implementation, the external inflatable cuff 107 is not inflated, and reflectance oximetry is used. Depicted in FIG. 3 is that, in one implementation, the “external anus” labeled striping 300 is positioned such that it is extensively coterminous with the pivot point 120 of the pulse oximetry assembly 102.

Furthermore, it is to be understood that the invention is solely defined by the appended claims. It will be understood by those within the art that, in general, terms used herein, and especially in the appended claims (e.g., bodies of the appended claims) are generally intended as “opera” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “d” and/or “ari” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

1. An anal pulse oximeter device comprising:
an anal canal surface; and
a rectal-vault cuff having a leading portion and a trailing portion, the trailing portion substantially proximate to said anal canal surface.

2. The anal pulse oximeter device of claim 1, wherein said anal canal surface comprises:
an anatomical anal canal surface; or
a surgical anal canal surface.

3. The anal pulse oximeter device of claim 1, wherein said anal canal surface comprises:
a bowel-evacuation channel such that a patient's bowel may be evacuated.

4. The anal pulse oximeter device of claim 1, wherein said anal canal surface comprises:
a cylindrically-shaped surface.

5. The anal pulse oximeter device of claim 1, wherein said anal canal surface comprises:
said anal canal surface formed from a medical-grade polymeric material.

6. The anal pulse oximeter device of claim 1, wherein said anal canal surface comprises:
either a light source or a light sensor substantially integral with said anal canal surface.

7. The anal pulse oximeter device of claim 6, wherein said light source or said light sensor substantially integral with said anal canal surface comprises:
a pulse oximetry adapter/plug operably coupled with either said light source or said light sensor.

8. The anal pulse oximeter device of claim 6, wherein said light source or said light sensor substantially integral with said anal canal surface comprises:
said light source or said light sensor either at, or movable to, a substantially non-zero angle relative to said anal canal surface.

9. The anal pulse oximeter device of claim 8, wherein said light source or said light sensor either at, or movable to, a substantially non-zero angle relative to said anal canal surface comprises:
a member adapted to hold either said light source or said light sensor at the substantially non-zero angle relative to said anal canal surface.

10. The anal pulse oximeter device of claim 9, wherein said member adapted to hold either said light source or said light sensor at the substantially non-zero angle relative to said anal canal surface comprises:
a member adapted to flex sufficient to hold either said light source or said light sensor at the substantially non-zero angle relative to said anal canal surface.

11. The anal pulse oximeter device of claim 9, wherein said member adapted to hold either said light source or said light sensor at the substantially non-zero angle relative to said anal canal surface comprises:
a member having curvature sufficient to hold either said light source or said light sensor at the substantially non-zero angle relative to said anal canal surface.

12. The anal pulse oximeter device of claim 8, wherein said light source or said light sensor either at, or movable to, a substantially non-zero angle relative to said anal canal surface comprises:
an external cuff adapted to hold either said light source or said light sensor at the substantially non-zero angle relative to said anal canal surface.

13. The anal pulse oximeter device of claim 1, wherein said anal canal surface comprises:
a physiological sensor substantially integral with said anal canal surface.

14. The anal pulse oximeter device of claim 13, wherein said physiological sensor substantially integral with said anal canal surface comprises:
a physiological sensor adapter/plug operably coupled with said physiological sensor.

15. The anal pulse oximeter device of claim 1, wherein said rectal-vault cuff comprises:
an inflatable cuff.

16. The anal pulse oximeter device of claim 1, further comprising:
an external cuff having a leading portion and a trailing portion, the leading portion substantially proximate to said anal canal surface.

17. The anal pulse oximeter device of claim 16, wherein said external cuff comprises:
an inflatable cuff.

18. The anal pulse oximeter device of claim 1, comprising:
rounded insertion end.

19. A method of using a pulse oximeter device comprising:
positioning an insertion end of the pulse oximeter device against an anus;
advancing the pulse oximeter device until a rectal-vault cuff of the pulse oximeter device substantially clears an internal anal sphincter; and
withdrawing the pulse oximeter device until the rectal-vault cuff of the pulse oximeter device contacts a rectal vault.

20. The method of claim 19, wherein said advancing the pulse oximeter device until a rectal-vault cuff of the pulse oximeter device substantially clears an internal anal sphincter comprises:
digitally verifying that the rectal-vault cuff of the pulse oximeter device has substantially cleared the patient's internal anal sphincter.

21. The method of claim 19, wherein said advancing the pulse oximeter device until a rectal-vault cuff of the pulse oximeter device substantially clears an internal anal sphincter comprises:
advancing the pulse oximeter device until an external anus marking is substantially proximate to the anus.

22. The method of claim 19, wherein said withdrawing the pulse oximeter device until the rectal-vault cuff of the pulse oximeter device contacts a rectal vault comprises:
withdrawing the pulse oximeter device until an increase in resistance is encountered.

23. The method of claim 19, wherein said withdrawing the pulse oximeter device until the rectal-vault cuff of the pulse oximeter device contacts a rectal vault comprises:
withdrawing the pulse oximeter device until a patient reports an anal sensation of contact.

24. The method of claim 19, further comprising:
detecting at least one physiological metric selected from the physiological-metric group including a pulse oximetry metric, a temperature metric, and a manometer metric.

25. The method of claim 19, further comprising:
evacuating a bowel via a bowel-evacuation channel of the pulse oximeter device.

26. A method of manufacturing a pulse oximeter device comprising:
attaching a rectal-vault cuff, the rectal-vault cuff having a leading portion and a trailing portion, such that the trailing portion is substantially proximate to an anal canal surface.

27. The method of claim 26, wherein the anal canal surface comprises:
an anatomical anal canal surface; or
a surgical anal canal surface.

28. The method of claim 26, wherein the anal canal surface comprises:
a bowel-evacuation channel such that a patient's bowel may be evacuated.

29. The method of claim 26, wherein the anal canal surface comprises:
a cylindrically-shaped surface.

30. The method of claim 26, wherein the anal canal surface comprises:
the anal canal surface formed from a medical-grade polymeric material.

31. The method of claim 26, wherein the anal canal surface comprises:
either a light source or a light sensor substantially integral with the anal canal surface.

32. The method of claim 31, wherein said light source or said light sensor substantially integral with said anal canal surface comprises:
a pulse oximeter adapter/plug operably coupled with said light source or said light sensor.

33. The method of claim 31, wherein said light source or said light sensor substantially integral with said anal canal surface comprises:
a member adapted to hold either said light source or said light sensor at a substantially non-zero angle relative to said anal canal surface.

34. The method of claim 31, wherein said light source or said light sensor substantially integral with said anal canal surface comprises:
a member adapted to flex sufficient to hold either said light source or said light sensor at a substantially non-zero angle relative to said anal canal surface.

35. The method of claim 31, wherein said light source or said light sensor substantially integral with said anal canal surface comprises:
a member having curvature sufficient to hold either said light source or said light sensor at a substantially non-zero angle relative to said anal canal surface.

36. The method of claim 31, wherein said light source or said light sensor substantially integral with said anal canal surface comprises:
an external cuff adapted to hold either said light source or said light sensor at a substantially non-zero angle relative to said anal canal surface.

37. The method of claim 26, wherein said anal canal surface comprises:
a physiological sensor substantially integral with said anal canal surface.

38. The method of claim 37, wherein said physiological sensor substantially integral with said anal canal surface comprises:
a physiological sensor adapter/plug operably coupled with said physiological sensor.

39. The method of claim 26, further comprising:
attaching an external cuff, the external cuff having a leading portion and a trailing portion, such that the leading portion is substantially proximate to said anal canal surface.
